(19) United States
(12) Patent Application Publication
(10) Pub. No.: US 2022/0397577 A1
(43) Pub. Date: Dec. 15, 2022

(54) Micro-Radiobinding Assays for Ligand Screening

(71) Applicant: AC Immune SA, Lausanne (CH)

(72) Inventor: Luigino Grasso, Lausanne (CH)

(73) Assignee: AC Immune SA, Lausanne (CH)

(21) Appl. No.: 17/642,800

(22) PCT Filed: Sep. 30, 2020
(86) PCT No.: PCT/IB2020/059171

§ 371 Date: Mar. 14, 2022

Related U.S. Application Data
(60) Provisional application No. 62/909,101, filed on Oct. 1, 2019.
(60) Provisional application No. 62/970,977, filed on Feb. 6, 2020.

(51) Int. Cl. G01N 33/60; G01N 33/68

(52) U.S. Cl. CPC G01N 33/60; G01N 33/6845

(57) ABSTRACT

The disclosure relates to binding assays that can measure the binding of ligands to a specific protein target in a micro-radiobinding assay. In particular, the present disclosure relates micro-radiobinding assays useful for low-abundance proteins, such as recombinant or tissue-derived proteins isolated from healthy or diseased, human donor samples.

### CROSS-REFERENCE TO RELATED APPLICATIONS

This application claims the benefit of priority of U.S. Provisional Application No. 62/909,101, filed Oct. 1, 2019, and U.S. Provisional Application No. 62/970,977, filed Feb. 6, 2020, each of which is incorporated by reference herein in its entirety for any purpose.

### FIELD

The present application relates to compositions and methods for a micro-radiobinding assay for ligand characterization and screening on proteins immobilized on a coated surface.

### BACKGROUND

Radiobinding assays aim at determining binding parameters that govern the interaction between a ligand and a target. Such assays can be used for different experimental paradigms, including saturation, competition, and kinetic binding experiments, to define distinct parameters of the ligand-target interaction.

There is a need for highly sensitive assays for low abundance biological targets.

### SUMMARY

Saturation assays aim to measure the affinity of a ligand to a target, referred as K_{d}. The K_{d} is the dissociation constant at equilibrium and is defined as the concentration of ligand necessary to occupy 50% of the binding sites of a given target. To determine the K_{d}, the target protein is incubated with a radiolabeled test ligand, where the protein is at a constant (fixed) concentration, while the concentration of the radiolabeled test ligand is varied. At equilibrium, the amount of bound ligand is quantified for each concentration of the radiolabeled test ligand until saturation occurs. The obtained data can be expressed as the amount of ligand bound to a target protein in molar concentration and therefore the K_{d} of the ligand can be calculated. The second type of radiobinding experiment is the competitive radiobinding assay. In the competitive format, the radiobinding assay measures the ability of a non-radiolabeled (cold) test ligand to displace a radiolabeled test ligand. The radiolabeled test ligand is used at a fixed concentration close to its K_{d}, and the non-radiolabeled test ligand is used at different concentrations so that an inhibitory (or displacement) constant (Ki) can be determined. This competition mode can also be used as a screening assay, in which one or multiple non-radiolabeled test ligands/test compounds are tested at single or multiple concentrations for their ability to displace one common radiolabeled tool ligand. Following the calculation of percentage of competition (if the test ligand is used at a single concentration) or Ki (if the test ligand is used at multiple concentrations), test ligands/test compounds can be ranked according to their potency in displacing the radiolabeled tool ligand. The ranking can be used to identify potent ligands for a defined target protein, and thus to drive discovery programs.

In a radiobinding assay, the radiolabeled ligand is labeled with a radioactive isotope and this allows the quantification of its bound fraction to the target. This is obtained by measuring the ligand intrinsic ionizing radioactivity with a detector containing photomultiplier devices. To estimate the amount of ligand that is bound to the target at equilibrium, the target-ligand complex (bound fraction of the ligand) needs to be separated from the unbound ligand (free fraction of the ligand). In a classical radiobinding assay, physical separation is usually accomplished by filtration, where the filter, generally made of nitrocellulose or glass fibers, retains only the bound ligand-target complex, while the free ligand passes through the filter and is removed. The bound fraction of the ligand can then be quantified. Classical filter-based radiobinding assays require large amounts of target protein to achieve the necessary protein concentrations in the large volumes required for the filtration processes. The need for a substantial protein amount limits the use of the assay, in particular when the target protein needs to be isolated from human tissue samples where it may be present at low levels.

The micro-radioligand binding assays described herein allow for characterization of binding of ligands to low-abundance proteins such as those derived from brain or other patient tissues or fluids. This includes, but is not limited to, proteins that are associated with neurodegenerative diseases. Indeed, these proteins are known to undergo conformational changes that lead to protein deposits and the accumulation of those proteinaceous deposits is directly linked to disease manifestation and progression. Examples of those proteins are: amyloid beta (Abeta) and tau, of which deposits are the hallmarks of Alzheimer's disease (AD), Down Syndrome and other tauopathies; alpha-synuclein (a-syn), of which deposits are the hallmark of Parkinson's disease (PD) and Dementia with Lewy Bodies; and TAR DNA-binding protein 43 (TDP-43), of which deposits are the hallmark of amyotrophic lateral sclerosis (ALS) and TDP-frontotemporal lobar degeneration (TDP-FTLD) (Serrano-Pozo et al., 2011, Spillantini et al., 1997, Neumann et al., 2006 and Nelson et al., 2019). These pathological protein deposits can be produced artificially in vitro from recombinant proteins, but it is widely recognized that the in vitro-produced deposits (such as aggregates) differ in conformation from protein isolated from patient tissues. Therefore, discovery programs that aim to target those protein deposits (such as aggregates) with therapeutic or diagnostic agents ideally would use brain-derived protein samples as targets for the pharmacological assays to ensure the generation of preclinical data with higher translational value.

The need of minimizing the amount of biological target required in traditional filter-based radiobinding assays led to the development of a microarray technique to investigate ligand binding to protein G protein coupled receptor (GPCR) isolated from cell lines (Posner et al., 2007). However, there remains a need for accurate, highly sensitive assay methods adapted for low abundance pathological proteins, for example, those derived from human brain samples.

The present application describes a miniaturized radiobinding assay specifically designed for low abundance protein targets, making it particularly suitable for pathological protein deposits derived from patient brain samples. The ability to screen compounds on human-derived, pathological protein deposits while minimizing the amount of patient-derived tissue required represents a major limitation of the commonly used filter-based radiobinding assay and a major advantage of the herein described micro-radiobinding assay. The micro-radiobinding assay allows for the use of very low amounts of protein targets, using up to 500-fold lower amount of protein target material than a classical filter-based radiobinding assay. This assay can be used to generate K_{d} and Ki values as well as a high-throughput assay for screening of ligand libraries. The assay was successfully validated by direct comparison with a classical, filter-based radiobinding assay.

The methods described herein use a microarray with localized microsamples of pathological protein on a coated surface. In some aspects, biochemically-enriched samples of pathological protein targets are spotted onto a coated surface (such as a coated glass surface) to form a pathological protein array with spots in well-defined positions. In some aspects, brain-derived protein samples are subjected to an enrichment step to concentrate the protein deposits (to ensure adequate signal from the assay) and to produce an enriched sample with suitable viscosity for proper dispensing or spotting on the coated surface. Detection of the signal is obtained by phosphor imaging, with the dried coated surface exposed to a phosphor imaging film or screen at the end of the different incubation steps. After exposure of the surface to the screen or film for an appropriate period of time, the screen is scanned with a phosphor imaging scanner and the signal quantified using an image analysis software, such as ImageJ-win 64 software.

In some aspects, the disclosure relates to a method of determining binding affinity (K_{d}) of a test ligand for a pathological protein in an enriched biological sample comprising:
contacting a plurality of aliquots of the enriched biological sample on a microarray with a cold test ligand at saturating fixed concentration;
contacting the aliquots with a radiolabeled test ligand at multiple concentrations to form a radiolabeled complex between the radiolabeled test ligand and the pathological protein in each aliquot;
removing unbound radiolabeled test ligand from the aliquots;
detecting a signal from the radiolabeled test ligand in the radiolabeled complex in each aliquot; and
calculating the K_{d} from the detected signals in each aliquot.

In some aspects, the disclosure relates to a method of determining binding affinity (K_{d}) of a test ligand for a pathological protein in an enriched biological sample comprising:
contacting a plurality of aliquots of the enriched biological sample on a microarray with a radiolabeled test ligand at multiple concentrations to form a radiolabeled complex between the radiolabeled test ligand and the pathological protein in each aliquot;
contacting the aliquots with a cold test ligand at saturating fixed concentration;
removing unbound radiolabeled test ligand from the aliquots;
detecting a signal from the radiolabeled test ligand in the radiolabeled complex in each aliquot; and
calculating the K_{d} from the detected signals in each aliquot.

In some aspects, the disclosure relates to a method of determining binding affinity (K_{d}) of a test ligand for a pathological protein in an enriched biological sample comprising:
contacting a plurality of aliquots of the enriched biological sample on a microarray with a radiolabeled test ligand at multiple concentrations and a cold test ligand at saturating fixed concentration to form a radiolabeled complex between the radiolabeled test ligand and the pathological protein in each aliquot;
removing unbound radiolabeled test ligand from the aliquots;
detecting a signal from the radiolabeled test ligand in the radiolabeled complex in each aliquot; and
calculating the K_{d} from the detected signals in each aliquot.

In some aspects, the disclosure relates to a method of determining the inhibitory constant (Ki) of a test ligand for a pathological protein in an enriched biological sample comprising:
contacting a plurality of aliquots of the enriched biological sample on a microarray with a radiolabeled test ligand at a fixed concentration close to the K_{d} for the test ligand to form a radiolabeled complex between the radiolabeled test ligand and the pathological protein in each aliquot;
contacting the aliquots with a cold test ligand at multiple concentrations;
removing unbound radiolabeled test ligand from the aliquots;
detecting a signal from the radiolabeled test ligand in the radiolabeled complex in each aliquot; and
calculating the Ki from the detected signals in each aliquot.

In some aspects, the disclosure relates to a method of determining the inhibitory constant (Ki) of a test ligand for a pathological protein in an enriched biological sample comprising:
contacting a plurality of aliquots of the enriched biological sample on a microarray with a cold test ligand at multiple concentrations;
contacting the aliquots with a radiolabeled test ligand at a fixed concentration close to the K_{d} for the test ligand to form a radiolabeled complex between the radiolabeled test ligand and the pathological protein in each aliquot;
removing unbound radiolabeled test ligand from the aliquots;
detecting a signal from the radiolabeled test ligand in the radiolabeled complex in each aliquot; and
calculating the Ki from the detected signals in each aliquot.

In some aspects, the disclosure relates to a method of determining the inhibitory constant (Ki) of a test ligand for a pathological protein in an enriched biological sample comprising:
contacting a plurality of aliquots of the enriched biological sample on a microarray with a radiolabeled test ligand at a fixed concentration close to the K_{d} for the test ligand and a cold test ligand at multiple concentrations to form a radiolabeled complex between the radiolabeled test ligand and the pathological protein in each aliquot;
removing unbound radiolabeled test ligand from the aliquots;
detecting a signal from the radiolabeled test ligand in the radiolabeled complex in each aliquot; and
calculating the Ki from the detected signals in each aliquot.

In some aspects, the disclosure relates to a method of evaluating a test compound for the ability to displace a radiolabeled tool ligand in a radiolabeled complex with a pathological protein in an enriched biological sample comprising:
contacting a plurality of aliquots of the enriched biological sample on a microarray with a radiolabeled tool ligand at a fixed concentration close to the K_{d} of the tool ligand to form a radiolabeled complex between the radiolabeled tool ligand and the pathological protein in each aliquot;
contacting the aliquots with a cold test compound at a single concentration or at multiple concentrations;
removing unbound radiolabeled tool ligand from the aliquots;
detecting a signal from the radiolabeled tool ligand in the radiolabeled complex in each aliquot; and
calculating (a) the percent of competition for the cold test compound from the detected signals in each aliquot, where the cold test compound is contacted at a single concentration; or (b) the Ki for the cold test compound from the detected signals in each aliquot, where the cold test compound is contacted at multiple concentrations.

In some aspects, the disclosure relates to a method of evaluating a test compound for the ability to displace a radiolabeled tool ligand in a radiolabeled complex with a pathological protein in an enriched biological sample comprising:
contacting a plurality of aliquots of the enriched biological sample on a microarray with a cold test compound at a single concentration or at multiple concentrations;
contacting the aliquots with a radiolabeled tool ligand at a fixed concentration close to the K_{d} of the tool ligand to form a radiolabeled complex between the radiolabeled tool ligand and the pathological protein in each aliquot;
removing unbound radiolabeled tool ligand from the aliquots;
detecting a signal from the radiolabeled tool ligand in the radiolabeled complex in each aliquot; and
calculating (a) the percent of competition for the cold test compound from the detected signals in each aliquot, where the cold test compound is contacted at a single concentration; or (b) the Ki for the cold test compound from the detected signals in each aliquot, where the cold test compound is contacted at multiple concentrations.

In some aspects, the disclosure relates to a method of evaluating a test compound for the ability to displace a radiolabeled tool ligand in a radiolabeled complex with a pathological protein in an enriched biological sample comprising:
contacting a plurality of aliquots of the enriched biological sample on a microarray with a cold test compound at a single concentration or at multiple concentrations and with a radiolabeled tool ligand at a fixed concentration close to the K_{d} of the tool ligand to form a radiolabeled complex between the radiolabeled tool ligand and the pathological protein in each aliquot;
removing unbound radiolabeled tool ligand from the aliquots;
detecting a signal from the radiolabeled tool ligand in the radiolabeled complex in each aliquot; and
calculating (a) the percent of competition for the cold test compound from the detected signals in each aliquot, where the cold test compound is contacted at a single concentration; or (b) the Ki for the cold test compound from the detected signals in each aliquot, where the cold test compound is contacted at multiple concentrations.

### BRIEF DESCRIPTION OF FIGURES

FIG. 1 shows a micro-radiobinding assay configuration. A solid surface (such as a glass slide coated with a hydrophobic adhesive surface such as an aminopropylsilane (APS) (A) is used as the support to spot the protein target. After spotting the surface with the protein using an automated spotting device, 64 pads of 9 spots each are obtained (B). In some embodiments, a surface with watertight chambers on the surface (C) is spotted manually with the protein target (D).

FIG. 2 show the results of binding affinity (K_{d}) determinations for a tool ligand on AD human brain derived tau deposits with a classical filter-based radiobinding assay (A) and a micro-radiobinding assay (B). The Y axis of FIG. 2A shows the measurement of the amount of specific radiolabeled ligand bound to the target expressed in counts per minutes (cpm). The Y axis of FIG. 2B represents the quantification of the intensity of the signal present on the film being proportional to the signal obtained with the amount of specific radiolabeled ligand bound to the target. The tool ligand compound showed a K_{d} of 11.8 nM by filter-based assay (A) and of 7.9 nM by micro-radiobinding assay (B). Both K_{d} had good fitting (R²=0.97 for (A) and R²=0.85 for (B)).

FIG. 3 show the results of binding constant (K_{d}) determinations for the test compounds on PD human brain-derived a-syn and Frontal Temporal Dementia (FTD) human brain-derived TDP-43 deposits using a micro-radiobinding assay (Compound 3, a-syn, A; Compound 2, a-syn, B; Compound 3, TDP-43, C). The Y axis of each figure represents the quantification of the intensity of the signal present on the film being proportional to the signal obtained with the amount of specific radiolabeled ligand bound to the target. Compound 3 showed a K_{d} of 10.8 nM on PD-brain derived a-syn with a good fitting (R²=0.87; A) and a K_{d} of 138 nM on FTD-brain derived TDP-43 with a good fitting (R²=0.79; C). Compound 2 showed a K_{d} of 7.8 nM on PD-brain derived a-syn with a good fitting (R²=0.80; B).

FIG. 4 show the results of the determination of displacement ability measured by Ki of a tritiated tool ligand on AD human brain-derived tau deposits (A), and of Compound 3 on PD human brain-derived a-syn deposits (B) using a micro-radiobinding assay. The Y axis of each figure represents the displacement of the labeled compound expressed in percentage, where 100% corresponds to complete displacement. The tool ligand showed a Ki of 1 nM with a good fitting (R²=0.97). Compound 3 showed a Ki of 41 nM with a good fitting (R²=0.84).

FIG. 5 show the results of screening of Compounds 4, 5, and 6 (A, B, and C, respectively) in the micro-radiobinding assay using a radiolabeled Compound 3 as a tool ligand. The Y axis of each figure represents the displacement of the labeled compound expressed in percentage, where 100% corresponds to complete displacement. Ki of compounds 4, 5 and 6 were measured at 13, 37 and 147 nM, all with good fitting (R²=0.97, 0.80 and 0.64 respectively).

### DETAILED DESCRIPTION

Additional aspects and advantages of the present disclosure will become apparent to those skilled in this art from the following detailed description, wherein illustrative aspects of the present disclosure are shown and described. As will be appreciated, the present disclosure is capable of other and different aspects, and its several details are capable of modifications in various respects, all without departing from the disclosure. Accordingly, the descriptions are to be regarded as illustrative in nature, and not as restrictive.

All publications, patents, and patent applications mentioned in this specification are herein incorporated by reference to the same extent as if each individual publication, patent, or patent application was specifically and individually indicated to be incorporated by reference.

A pathological protein is a protein that produces pathological effects upon abnormal accumulation in human tissues or bodily fluids. In some embodiments, pathological proteins are proteins that form deposits, such as filaments, tangles, or other aggregates, upon such accumulation, and the deposits cause dysfunction and disease progression. In some embodiments, the pathological proteins used in the assays described herein are produced through methods known to the person skilled in the art. In some embodiments, the pathological proteins used herein are derived from human biological samples. In some embodiments, the pathological protein is present in a human biological sample, which is enriched through methods known to the person skilled in the art to provide a more concentrated biological sample for use in the assays described herein. In some embodiments, the enriched biological sample comprises from about 1 to about 6.5 mg/mL of total protein (pathological protein target plus other sample proteins). In some embodiments, the enriched biological sample comprises from about 1 to about 2 mg/mL of total protein (pathological protein target plus other sample proteins). In some embodiments, the enriched biological sample comprises from about 3.5 to about 6.5 mg/mL of total protein (pathological protein target plus other sample proteins). In some embodiments, the enriched biological sample also comprises lipids, RNA, DNA, or other cellular components.

In some embodiments, the human biological sample is a human body fluid (such as a nasal secretion, a urine sample, a blood sample, a plasma sample, a serum sample, an interstitial fluid (ISF) sample or a cerebrospinal fluid (CSF) sample) or a human tissue sample (e.g., derived from heart, muscle, brain, etc., tissue). In other embodiments, the human biological sample is a blood sample or a cerebrospinal fluid sample. In some embodiments, the human biological sample is a brain sample, such as a brain cortex sample or a hippocampus sample. In some embodiments, the pathological protein is associated with a neurodegenerative disease. In some embodiments, the enriched biological sample is derived from a human biological sample from a patient suffering from or a deceased patient who suffered from a neurodegenerative disease. In some embodiments, the neurodegenerative disease is Alzheimer's disease, Down Syndrome, Parkinson's disease, fronto-temporal dementia, amyotrophic lateral sclerosis, Dementia with Lewy Bodies, progressive supranuclear palsy (PSP), Multiple System Atrophy (MSA), or traumatic brain injury, limbic-predominant age-related TDP-43 encephalopathy (LATE), Chronic Traumatic Encephalopathy (CTE). In some embodiments, the pathological protein is Tau, Abeta, α-synuclein, Inflammasome component (including but not limited to ASC), Dipeptide Repeat (DPRs) derived from C9orf72 or TDP-43. In a preferred embodiment, the pathological protein is Tau, Abeta, α-synuclein, or TDP-43.

In some embodiments, a microarray is prepared by dispensing aliquots of an enriched biological sample onto a solid support in a repeating pattern. In some embodiments, the aliquots are dispensed onto the solid support. In some embodiments, the aliquot is a spot on the solid support. Thus, in some embodiments, the methods described herein further comprise preparing the microarray by dispensing aliquots of the enriched biological sample onto a glass slide. In some embodiments, the aliquot of enriched biological sample is substantially dried on the microarray. In some embodiments, the microarray comprises at least 25, or at least 50, or at least 100, or at least 200, or at least 300, or at least 400, or at least 500 spots, or from 250 to 600 spots, or from 500 to 600 spots. In some embodiments, the spots are grouped in pad profiles comprising at least 1, 2, 3, 4, 5, 6, 7, 8, 9, or 10 spots, or from 4 to 10 spots, or 9 spots. In some embodiments, the microarray solid support is divided into chambers, with each chamber comprising a pad profile defined as the number of spots in the chamber. In some embodiments, the discrete chambers are configured so that different fluids or reagents can be added to each individual chamber without mixing between chambers. In some embodiments, the microarray comprises at least 2, or at least 5, or at least 10, or at least 15, or at least 20, or at least 25, or at least 30, or at least 40, or at least 45, or at least 50, or at least 55, or at least 60, or about 64 chambers. In some embodiments, different known concentrations of test ligand are used at different aliquots, spots, pad profiles, or chambers in the microarray. In some embodiments, contacting the aliquots with multiple concentrations comprises contacting each chamber in the microarray with a different concentration. Where each chamber comprises multiple aliquots or spots, those aliquots or spots serve as replicates for the test conditions (e.g., test compound or test concentration) for that chamber.

In some embodiments, the dispensing of aliquots, or spotting, of the pathological protein from the enriched biological sample is done using a spotting device, such as an automated spotting device (e.g. Nano-Plotter), or by manual pipetting. In some embodiments, spotting is performed using a Nano-Plotter 2.1™ (GESIM; Germany). In some embodiment of the invention, the volume of the enriched biological sample comprising the pathological protein that is arrayed is at least 300 picoliters, or at least 1 nanoliter, or at least 10 nanoliters, or at least 36 nanoliters, or is a volume in the range of about 200 picoliters to about 36 nanoliters, or about 200 picoliters to about 10 nanoliters, or about 200 picoliters to about 1 nanoliter.

The microarray comprises a coated solid support and the solid support can be any suitable solid material, such as glass or a polymer. In some embodiments, the solid support is a glass slide. In some embodiments, the microarray solid support is coated with an adherent. In some embodiments, the adherent is a silane, a thiol, a disulfide, an epoxide, and/or a polymer. In some embodiments, the adherent is a silane. In some embodiments, the adherent is an aminopropylsilane. In some embodiments, the microarray solid support is an aminopropylsilane-coated glass slide.

A ligand, tool ligand, test compound or test ligand is an organic compound, an antigen, an antibody, a peptide, a protein, or a protein captured by an antibody. In some embodiments, a ligand, tool ligand, test compound or test ligand is an organic compound, such as a chemical compound or a small molecule compound. In some embodiments, the tool ligand and test ligand are both small molecule compounds. In some embodiments, the tool ligand and test compound are both small molecule compounds.

A labeled ligand, radiolabeled ligand, labeled tool ligand, radiolabeled tool ligand, labeled test ligand, labeled test compound, radiolabeled test compound, or radiolabeled test ligand is an organic compound, antigen, antibody, peptide, protein, or protein captured by an antibody comprising a label that allows for quantification of the ligand, tool ligand, test compound, or test ligand. In some aspects, the label allows for quantification of the amount of ligand, tool ligand, test compound, or test ligand bound to a pathological protein. The type of the label is not specifically limited and will depend on the detection method chosen. The position at which the detectable label is to be attached to the ligands of the present invention is not particularly limited.

In some embodiments, the radiolabeled test ligand is a radiolabeled version of the test ligand. In some embodiments, the radiolabeled tool ligand is a radiolabeled version of a known ligand. In some embodiments, the tool ligand or radiolabeled tool ligand is a known ligand for the pathological protein of interest. Exemplary radiolabeled tool ligands include: Abeta ([11C]PiB (Pittsburgh Compound B), [18F]florobetapir, [18F]florobetaben, or [18F]flutematamol); Tau ([18F]T-807 (also known as AV1451), flortaucipir [18F]MK-6240, [18F]RO6958948, [18F]PI-2620, [18F]-GTP-1, [18F]JNJ-067, [18F]PM-PBB3, or [11C]PBB3), THK-5351, THK-5562; or Alpha-synuclein ([3H]SIL26). Exemplary tool ligands include unlabeled versions of these exemplary radiolabeled tool ligands.

Exemplary labels include isotopes such as radionuclides, positron emitters, or gamma emitters, as well as fluorescent, luminescent, and/or chromogenic labels. Radioisotopic labels, as used herein, are present in an abundance that is not identical to the natural abundance of the radioisotope. Furthermore, the employed amount should allow detection thereof by the chosen detection method. In some embodiments, the label is a radionuclide label. Examples of suitable isotopes as radionuclides include ²H, ³H, ¹⁸F, ¹²³I, ¹²⁴I, ¹²⁵I, ¹³¹I, ¹¹C, ¹³N, ¹⁵O, and ⁷⁷Br. In some embodiments, the radionuclide label is ²H, ³H, ¹¹C, ¹³N, ¹⁵O, or ¹⁸F. In some embodiments, the radionuclide label is ²H, ³H and ¹⁸F. In some embodiments, the radionuclide label is ³H. Radiolabeled compounds as described herein are generally be prepared by conventional procedures known to the persons skilled in the art using appropriate isotopic variations of suitable reagents, which are commercially available or are prepared by known synthetic techniques.

The tool ligands, radiolabeled tool ligands, test ligands, test compounds, and radiolabeled test ligands can also be provided in the form of a composition with one or more of a blocking agent, diagnostically acceptable carrier, diluent, excipient, or buffer. In some embodiments, the composition comprises a blocking agent. In some embodiments, the blocking agent is bovine serum albumin (BSA), casein, or albumin from chicken egg white. In some embodiments, the blocking agent is BSA. A blocking agent blocks non-specific binding sites on the pathological protein and reduces background signal. In some embodiments, the methods comprise treating the aliquots of the enriched biological sample with a blocking agent prior to or simultaneously with the first contacting of the aliquots. In some embodiments, treating the aliquots with a blocking agent comprises treating the aliquots with an assay buffer comprising the blocking agent, optionally where the assay buffer comprises Tris-HCl or phosphate-buffered saline (PBS).

As used herein, “saturating fixed concentration” means the concentration that saturates specific binding for a particular protein.

As used herein, contacting aliquots on a microarray with “multiple concentrations” of a ligand or compound means contacting different aliquots or sets of aliquots with different concentrations of the ligand or compound. Where a set of aliquots on the microarray is contacted with a given concentration, the aliquots in that set serve as replicates for the test concentration. An aliquot or set of aliquots may be segregated from other aliquots or sets of aliquots on a microarray in, for example, individual chambers. For methods involving determining binding affinity, in some embodiments, a suitable range of test concentrations is at least 50-fold lower relative to the saturating fixed concentration.

In methods of determining the inhibitory constant of a test ligand, the aliquots are contacted with a radiolabeled test ligand “at a fixed concentration close to the Kd for the test ligand,” which refers to within about 2-fold of the Kd.

In some embodiments, removing unbound ligand (e.g., test ligand, test compound, or radiolabeled ligand) comprises washing the microarray to remove ligand that is not bound to the protein target (unbound ligand). In some embodiments, washing comprises washing with a buffer. In some embodiments, the buffer is PBS.

In some embodiments, detecting comprises detecting a signal on a film after exposing a microarray comprising a complex comprising a radiolabeled tool ligand or radiolabeled test ligand to the film. In some embodiments, the film is a phosphoscreen film. Quantification of signals according to some embodiments are realized by scanning, or by photoimager software such as Phosphoimager Typhoon IP. Images can be quantified by using image analysis software, such as ImageJ-win 64 software. In some embodiments, detecting comprises exposing the microarray comprising the radiolabeled test or tool ligand to a film, such as a phosphoscreen film, thereby generating a signal on the film, and quantifying the signal on the film. In some embodiments, detecting comprises measuring the radioactivity signal (number of disintegrations) by exposing a microarray comprising a complex comprising a radiolabeled tool ligand or radiolabeled test ligand to a real-time autoradiography system based on a new generation of gas detectors (e.g. BeaQuant instrument [ai4R], BetaIMAGER, [Biospace Lab]). Quantification of signals according to some embodiments are performed by digital imaging. In some embodiments, images can be quantified by using the image analysis software (Beamage [ai4R], M3 vision [Biospace Lab]). In some embodiments, images can be exported to an image processing tool and can be quantified by using image analysis software, such as ImageJ-win 64 software.

In some embodiments is a method comprising:
spotting a pathological protein on a glass support in a pad profile, for example, on an aminopropylsilane (APS) coated glass slides;
contacting the spotted protein with a non-labeled (cold) ligand to form a complex between the ligand and the protein;
contacting the complex with a labeled ligand to form a labeled complex between the labeled ligand and the protein;
washing the labeled complex with a buffer, for example, a PBS buffer;
drying the glass support, for example, at room temperature or under an argon-flow; exposing the glass support to a film, for example a phosphoscreen film; and
quantifying the signal on the film after exposure of the labeled ligand bound to the protein.
   In some embodiments, the spotted protein is contacted with a blocking agent. In some such embodiments, the blocking agent is present in an assay buffer with the cold ligand and/or in an assay buffer with the labeled ligand.

In some embodiments, the method comprises quantifying the signal on the film after exposure of the labeled ligand bound to the protein and determining the value of the binding affinity (K_{d}), for example by plotting the quantified values on a graph, such as by plotting the values on a graph by using an image software analysis.

In some embodiments is a method comprising: spotting a pathological protein on a glass support organized in a pad profile, particularly on a aminopropylsilane (APS) coated glass slides; bringing a composition comprising a labeled ligand in contact with the spotted protein; allowing the labeled ligand to form a complex with the protein; bringing a composition comprising a non-labeled (cold) ligand in contact with the complex comprising the protein and the labeled ligand; washing with a buffer, such as a PBS buffer; drying the glass support, such as the APS-coated glass slides, optionally at room temperature or under an argon-flux; exposing the glass support, such as the APS-coated glass slide, to a film, such as a phophoscreen film; quantifying the signal on the film after exposure of the labeled ligand bound to the protein; and determining the inhibition constant (Ki), preferably by plotting the quantified signal on a graph more preferably by plotting the quantified values on a graph by using an image software analysis.

In some embodiments is a method comprising: spotting a pathological protein on a glass support organized in a pad profile, such as on a aminopropylsilane (APS) coated glass slides, bringing a composition comprising a labeled ligand in contact with the spotted pathological protein and allowing the labeled ligand to form a complex with the protein; bringing a composition comprising a non-labeled ligand in contact with the complex comprising the protein and the labeled ligand; washing with a buffer, such as PBS; drying the glass support, optionally at room temperature or under an argon-flux; exposing the glass support to a film, such as a phosphoscreen film; quantifying the signal on the film after exposing the labeled ligand bound to the protein; and determining the inhibition ability (inhibitory constant, Ki), such as by plotting the quantified signal on a graph, or by plotting the quantified value on a graph by using an image software analysis. In some embodiments, the steps comprised before the drying are repeated at least 6 times, or at least 8 times, or at least 12 times. In some embodiments, the amount of ligand is increasing/decreasing each time the steps are repeated. In some embodiments, a Ki value is used to evaluate whether the compound has a capacity of competing with the binding of the labeled ligand to the protein. In some embodiments, a Ki value is used to rank the tested compounds according to their Ki values.

Also disclosed herein are kits for use in screening or evaluating test ligands/test compounds for their capability of binding a target or to for their capability of competing with the binding of a labeled ligand to a target. Such kits comprise components for performing the methods described herein, such as, for example, buffers, detectable dyes, laboratory equipment, reaction containers, instructions and the like.

In some embodiments, the disclosure provides for an assay to determine the binding affinity (K_{d}) of a test ligand/test compound for a pathological protein target. In other embodiments, the disclosure provides for an assay to determine the inhibitory constant (Ki) for a test ligand/test compound for a pathological protein target. In some aspects, the disclosure provides an assay for evaluation, selection, and/or screening of a test ligand/test compound or a series of test ligands/test compounds, wherein a test ligand/test compound is selected or the test ligands/test compounds are ranked according to the assay results.

In some methods of evaluating or screening a test compound for the ability to displace a radiolabeled tool ligand in a radiolabeled complex with a pathological protein in an enriched biological sample, the method comprises:

(a) contacting the aliquots with multiple cold test compounds each at a single concentration, or
(b) contacting the aliquots with multiple cold test compounds at multiple concentrations.
In some embodiments, the method comprises ranking the multiple test compounds according to the calculated percent of competition or Ki for each test compound. In some embodiments, multiple cold test compounds is at least two, at least five, at least 10, at least 25, at least 50, or at least 100 cold test compounds, or from two to 100, or from five to 100, or from 10 to 100, or from 25 to 100, or from 50 to 100 cold test compounds.

In any of the methods described herein, contacting the coated surface spotted with a plurality of aliquots of the enriched biological sample with a non-radiolabeled ligand may occur before, simultaneously with, or after contacting the coated surface with a radiolabeled ligand.

### EXAMPLES

The following examples are included to further describe some embodiments of the present disclosure and should not be used to limit the scope of the disclosure. The examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

While aspects of the present disclosure have been shown and described herein, it will be apparent to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the disclosure. It is intended that the following claims define the scope of the disclosure and that methods and structures within the scope of these claims and their equivalents be covered thereby.

### Example 1: Preparation of Proteins for Microarray Fabrication

### a) AD Brain-Derived Pathological Tau Protein

AD brain derived Tau paired-helical filaments (PHF) were enriched from the post-mortem brain of one Alzheimer's disease (AD) patient obtained from an external source (Tissue Solutions, UK). The enrichment procedure was modified from Jicha et al., 1997, and Rostagno and Ghiso, 2009, and was adapted from Spillantini et al., 1998, which described the extraction of dispersed a-syn filaments from brain of PD cases applying a procedure that was originally developed for the extraction of dispersed paired helical and straight filaments from Alzheimer's disease brain (Greenberg, S. G. et al., 1990; Goedert, et al., 1992). Briefly, the tissue was homogenized at a 1:4 ratio weight per volume ratio of tissue homogenization buffer volume [0.75 M NaCl in RAB buffer (100 mM 2-(N-morpholino)ethanesulfonic acid (MES), 1 mM EGTA, 0.5 mM MgSO₄, 2 mM DTT, pH 6.8) supplemented with protease inhibitors (Complete; Roche 11697498001)] in a glass Dounce homogenizer. The homogenate was then incubated at 4° C. for 20 min to let depolymerize any residual microtubules, before being transferred into polycarbonate centrifuge bottles (16×76 mm; Beckman 355603) and centrifuged at 11,000 g (12,700 RPM) in an ultracentrifuge (Beckman, XL100K) for 20 min at 4° C. using the pre-cooled 70.1 rotor (Beckman, 342184). Pellets were kept on ice. Supernatants were pooled into polycarbonate bottles and centrifuged again at 100,000 g (38,000 RPM) for 1 hour at 4° C. in the 70.1 Ti rotor to isolate PHF-rich pellets, whereas soluble Tau remained in the supernatants. The pellets from the first and second centrifugations were resuspended in 120 mL of extraction buffer [10 mM Tris-HCl pH 7.4, 10% sucrose, 0.85 M NaCl, 1% protease inhibitor (Calbiochem 539131), 1 mM EGTA, 1% phosphatase inhibitor (Sigma P5726 and P0044)]. The solution was then transferred into polycarbonate centrifuge bottles (16×76 mm; Beckman 355603) and centrifuged at 15,000 g (14,800 RPM) in an ultracentrifuge (Beckman, XL100K) for 20 min at 4° C. using the 70.1 Ti rotor. In the presence of 10% sucrose and at low speed centrifugation, most PHF remained in the supernatant whereas intact or fragmented NFTs and larger PHF deposits/aggregates were pelleted. The pellets were discarded. 20% Sarkosyl (Sigma L7414-10 ML) was added to the supernatants to a final concentration of 1% and stirred at room temperature for 1 hour. This solution was then centrifuged in polycarbonate bottles at 100,000 g (38,000 RPM) for 1 hour at 4° C. in the 70.1 Ti rotor, and the pellets containing PHF-rich material were resuspended in PBS in a 1:0.1 weight per volume ratio of tissue of PBS, aliquoted and stored at −80° C. Samples were analyzed for tau by western blot.

b) PD Brain-Derived a-Syn Protein

The procedure was adapted from the protocol described in Spillantini et al., 1998. Frozen tissue blocks from either temporal cortex or amygdala brain regions were thawed on ice and white matter was removed using a scalpel. The tissue was homogenized at a 1:4 weight per volume ratio of tissue to homogenization buffer volume using a glass dounce homogenizer. For homogenization, RAB buffer (100 mM 2-(N-morpholino)ethanesulfonic acid (MES), 1 mM EGTA, 0.5 mM MgSO₄, 2 mM DTT, pH 6.8) containing 0.75 mM NaCl and 1× protease inhibitors (Complete; Roche 11697498001) was used. The homogenate was then incubated at 4° C. for 20 minutes to allow depolymerization of any residual microtubules, before being transferred into polycarbonate centrifuge bottles (16×76 mm; Beckman 355603) and centrifuged at 11,000 g (12,700 RPM) in an ultracentrifuge (Beckman, XL100K) for 20 minutes at 4° C. using a pre-cooled 70.1 rotor (Beckman, 342184). Pellets were kept on ice while supernatants were pooled into polycarbonate bottles and centrifuged again at 100,000 g (38,000 RPM) for one hour at 4° C. in a 70.1 Ti rotor to separate a-syn deposits/aggregates from soluble a-syn. The pellets from the first and second centrifugations were resuspended in extraction buffer at 1:10 (weight per volume, w/v) ratio [10 mM Tris-HCl pH 7.4, 10% sucrose, 0.85 mM NaCl, 1% protease inhibitor (Calbiochem 539131), 1 mM EGTA, 1% phosphatase inhibitor (Sigma P5726 and P0044)]. The solution was then transferred into polycarbonate centrifuge bottles (16×76 mm; Beckman 355603) and centrifuged at 15,000×g (14,800 RPM, a 70.1 Ti rotor) for 20 minutes at 4° C. Pellets were discarded and sarkosyl (20% stock solution, Sigma L7414) was added to the supernatants to a final concentration of 1% and stirred at room temperature for one hour. This solution was then transferred to polycarbonate bottles and centrifuged at 100,000 g (38,000 RPM, 70.1 Ti rotor) for one hour at 4° C. Pellets containing enriched a-syn deposits/aggregates were resuspended in PBS in a 1:0.1 weight per volume ratio of tissue aliquoted and stored at −80° C. until use. The final fraction obtained by the procedure was analyzed biochemically (e.g., AlphaLISA, Western blot, and dot blot) with antibodies against a-syn to confirm the enrichment of a-syn deposits/aggregates.

### c) Frontotemporal Dementia (FTD) Brain-Derived TDP-43 Proteins

A section of brain tissue (cortex) from TDP-43 pathology human brain was cut with a scalpel in P2 lab and the tissue was weighed on Petri dishes. The tissue was transferred with tweezers to 2 ml homogenization tubes (CKmix). Homogenization buffer containing protease inhibitors was added to the dissected tissue at a 1:4 (w/v) ratio resulting in 20% brain homogenates. The suspension was homogenized at 4° C. with precellys using the following program: 3×30 sec at 5000 rpm, pause—15 sec between each cycle. The homogenized tissue was pooled and resuspended in a 5 ml Eppendorf tube. Aliquots of 600 μl of the homogenized brain were prepared and frozen on dry ice and stored at −80° C. Solubilization was performed in 1.5 mL protein low binding tubes (Eppendorf).

Brain homogenates were thawed on ice and resuspended in HS buffer to a final concentration of 2% Sarkosyl, 1 unit/μL Benzonase, and 1 mM MgCl₂ and were incubated at 37° C. under constant shaking at 600 rpm on a thermomixer for 45 min. Supernatants were collected in new tubes. The pellets were resuspended in 1000 μl myelin floatation buffer and centrifuged at 20,000 g for 60 min at 4° C. on the benchtop centrifuge. The supernatant was carefully removed with 1000 μl tip to remove all the floating lipids. Resuspension, centrifugation, and supernatant removal are repeated if lipids cannot be removed in a single centrifugation step. The resulting pellet was washed with PBS and centrifuged for 30 min at 4° C. on the benchtop centrifuge. The pellet was then resuspended in 200 μl PBS. All the enriched material was pooled and frozen at −80° C.

Samples were analyzed by western blot (phosphorylated TDP-43, TDP-43, Histone H3, Aβ).

### Example 2: Preparation of Microarrays for Pathological Proteins

### a) Method 1—Automated Spotting

Protein samples were diluted 1:3 (V/V) in PBS or assay buffer (50 mM Tris-HCl pH 7.5 in 0.9% NaCl, 0.1% BSA) and were homogenized by pipetting with P200 (Eppendorf) in an eppendorf 1.5 ml tube. Samples were then ready for automatic spotting onto aminopropylsilane (APS)-coated 64-pad microarray glass slides (Lucerna-Chem, #63475) using an automated spotting device, non-contact piezoelectric printer Nano-Plotter 2.1 (GeSiM; Germany). The automated spotting device is a versatile non-contact array printer that allows dispensing tiny volumes (picoliters to nanoliters) of liquid with an electrical pulse.

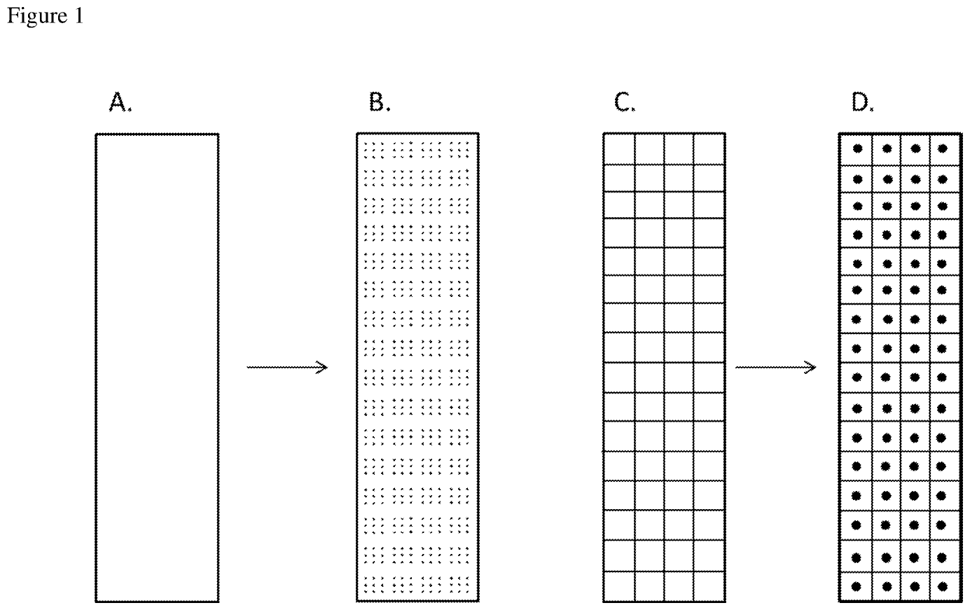

APS glass slides (FIG. 1A) were manually placed on the automatic trail and checked for their proper fixation to ensure high reproducibility of spotting between slides and good positioning of the drops when each glass slide was mounted with the chamber. The appropriate volume was pipetted from the loading plate using the piezoelectric tips. The system was optimized to allow dispension of 12×3 nL drops per spot, with 9 spots per pad and a total of 64 pads on each slide (FIG. 1B). Spotting of samples was performed in a humidity-controlled atmosphere at 65% relative humidity. Homogenization quality of the dispensed drops was assessed prior spotting to ensure that the volume and the density of the sample were constant throughout the dispensing. To do so, each drop was measured as it was dispensed out of the tip and dispersion of the drop was measured under a specific voltage. Once the slide was spotted, a chamber was assembled with Proplate mutiwell chambers 64 wells (25×75 mm glass microscope glass), and watertightness of the compartments was ensured using 2 ProPlate® Clips made of stainless steel on both sides of the glass slide. The system had 64 independent wells. Samples were left to dry for 15 minutes in the humidified chamber and subsequently were stored at 4° C. until use.

### b) Method 2—Manual Spotting

Protein samples were spotted manually by pipetting 1 μL with a micropipette p2 (Eppendorf) onto a glass slide with mounted chambers (FIGS. 1C and 1D). Only one drop was pipetted at each location and one drop corresponded to a spot on the glass slide. The resulting glass slides were dried at room temperature in a classical laboratory hood for at least 2 hours.

### Example 3: Preparation of Cold Samples

Cold compounds (test ligands or test compounds) were resuspended as a stock solution at 2.5 or 10 mM in 100% DMSO. Dilutions of cold compounds were obtained by performing a serial dilution series of 12 points, with a dilution factor of 2 to 3. Dilutions were performed in 100% DMSO to ensure a constant concentration of final DMSO concentration of 1% to 2.5% in the binding assay reaction volume. The maximal concentration of the cold compound used was 2 or 3 μM depending on the target, and that condition was also used for determining maximal displacement of the signal.

### Example 4: Preparation of Labeled Samples

Labeled compounds (radiolabeled test ligands or radiolabeled tool ligands; 1 mCi/mL) were synthesized and dissolved in 100% ethanol. Labeled compounds were diluted into the assay buffer to appropriate concentrations in series of concentrations in experiments to determine Kd or at a constant fixed concentration in experiments used to assess displacement potency.

### Example 5: Determination of Tau Binding Affinity (K_{d}) by Micro-Radiobinding Assay

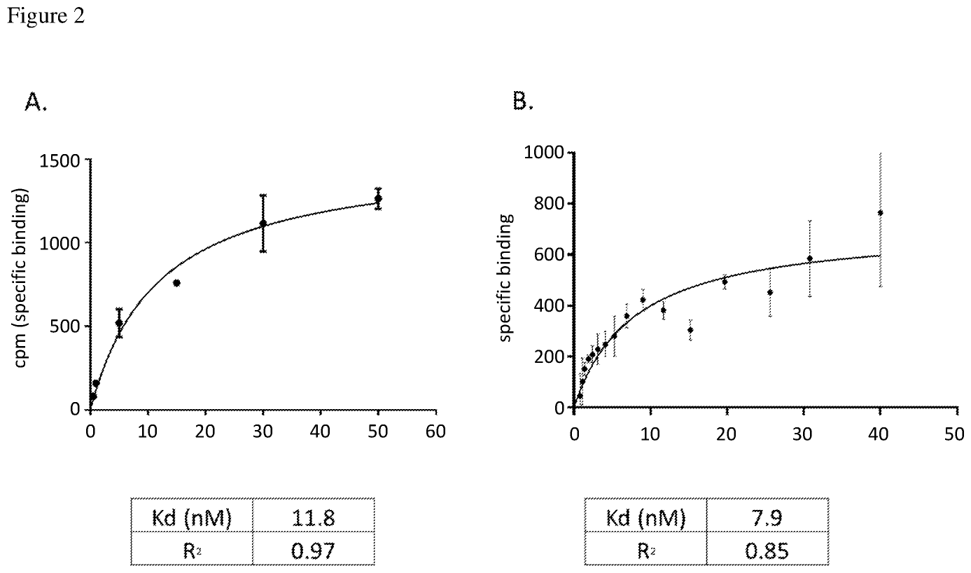

Chambers with spotted pathological Tau protein samples were mounted and filled with assay buffer (50 mM Tris pH: 7.5, 138 mM NaCl, 0.1% BSA) containing cold test ligand at 2 μM. The chambers were incubated for 120 min at room temperature. A sealing film was used to avoid evaporation. An equal volume of tritiated test ligand in assay buffer at varying concentrations was added to each chamber, mixed well, and incubated at room temperature. The final reaction volume was 40 μL. After 60 min of incubation, the reaction solution containing radioactive substances was collected in a suitable receptacle. The chambers were washed five times with ice-cold wash buffer. The ProPlate® chamber was disassembled from the glass slide and the glass slide was washed with double-distilled H₂O. Glass slides were dried under Argon flux under a chemistry hood. Films were exposed for at least 3 days on BAS-IP TR 2025 fujifilm in a Hypercassette (Amersham, RPN 11643). Films were scanned with a Phosphoimager Typhoon IP with a resolution of 50 μm and a sensitivity of 4000. Images were then analyzed and quantified using ImageJ-win 64 software. Graphs were generated using GraphPad Prism 7.03. A K_{d} of 7.9 nM with a good fit for the tool ligand with Tau deposits/aggregates was determined (FIG. 2B).

### Example 6: Comparison of Micro-Radiobinding Assay to Filter-Based Radiobinding Assay

A direct comparison of K_{d} determination using this classical filter-based assay and the micro-radiobinding assay described above was performed to assess the differences between the methods. To perform the filter-based assay, AD brain derived Tau was diluted 1/80 and was incubated with tritiated test ligand (a known Tau binder) at concentrations ranging from 1 to 50 nM and with or without the cold test ligand at a constant (fixed) concentration of 2 μM for 120 minutes at 25° C. A volume of 35 μL of each sample was filtered under vacuum on a GF/C filter plate (PerkinElmer 6005174) to trap the AD brain derived Tau with the bound test ligand, and the GF/C filters were washed three times with Tris 50 mM buffer pH 7-5. The GF/C filters were then vacuum-dried, 50 μL scintillation liquid (Ultimate Gold MB, PerkinElmer) was added to each well, and the filters were analyzed on a Microbeta2 device. Non-specific signal was determined with the sample containing the excess of cold test ligand (2 μM) and specific binding was calculated by subtracting the non-specific signal from the total signal. All measurements were performed with at least two technical replicates. The K_{d} value was calculated by nonlinear regression, one site specific binding using Prism V7 (GraphPad), to provide a K_{d} of 11.8 nM (FIG. 2A). Using the same test ligand, it was shown that very similar binding affinity values for independent AD brain-derived Tau deposits/aggregates were obtained using the two methods (11.8 nM (FIG. 2A) vs. 7.9 nM (FIG. 2B, described in Example 5)). The results validate the micro-radiobinding assay method as a robust alternative to the classical filter-based radiobinding assay.

### Example 7: Determination of K_{d} for a-Syn and TDP-43 with Micro-Radiobinding Assay

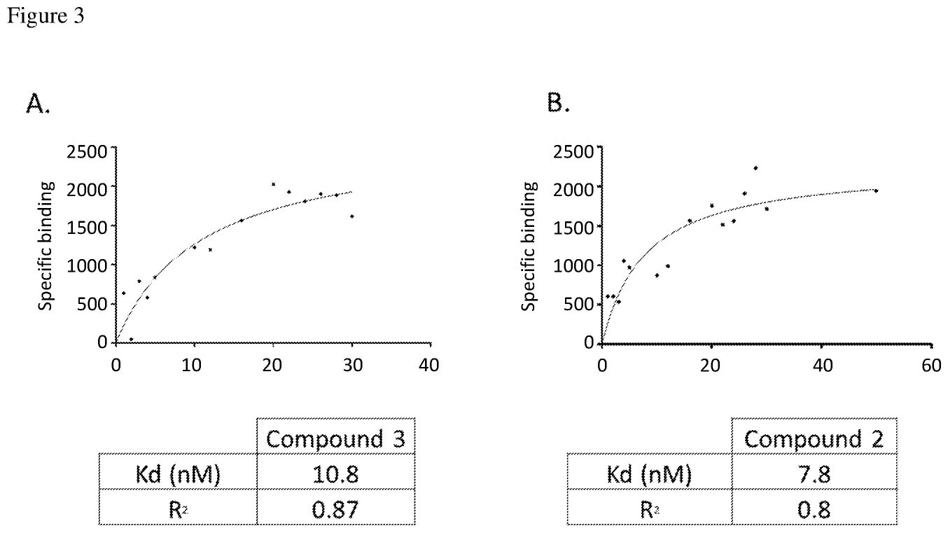

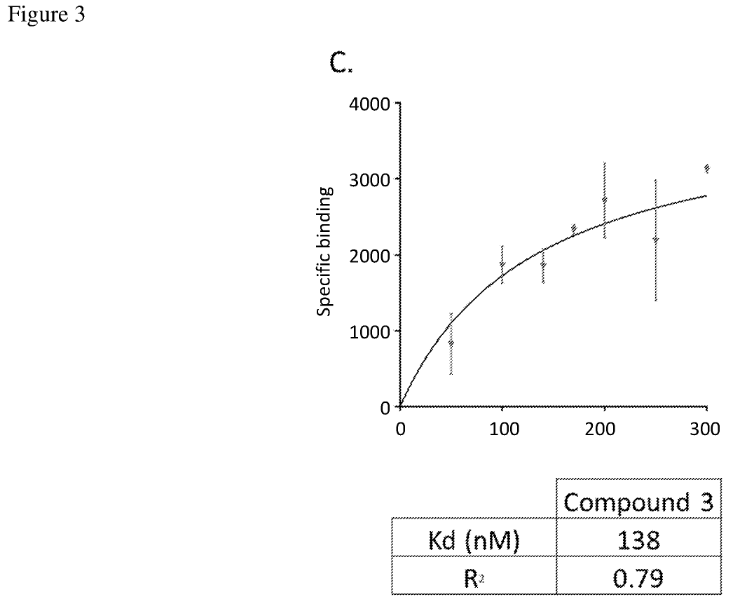

The method described in Example 5 was also used to determine the binding constant (K_{d}) of test ligands (Compound 2 (see PCT Appln. No. WO2019234243) and Compound 3) to protein targets a-syn (for Compound 2 and 3) and TDP-43 (for Compound 3). TDP-43-enriched fractions isolated from FTD brain or a-syn-enriched fractions isolated from PD brain were incubated with increasing concentrations (1 to 300 nM or 1 to 30 nM, respectively) of radio-labeled [³H] Compound 3 with or without a constant amount of cold Compound 3 at 2 PM. Similarly, a-syn-enriched fraction isolated from an PD brain was incubated with increasing concentrations (1 to 30 nM) of radio-labeled [³H] Compound 2 with or without a constant amount of cold Compound 2 at 2 μM. A constant excess concentration of cold Compound 2 (2 μM) or cold Compound 3 (2 μM) was used to determine nonspecific binding. K_{d} values of 10.8 (FIG. 3A) and 138 nM (FIG. 3C), respectively, were determined for Compound 3 for a-syn and TDP-43 proteins, and a K_{d} of 7.8 nM was determined for Compound 2 for a-syn (FIG. 3B). The results demonstrate that K_{d} values can be determined by the described micro-radiobinding assay for several target proteins that are known to be present in biological tissues in low or relatively low abundance. For example, pathological a-syn and pathological TDP-43 are considered to be present in lower abundance than pathological Tau in the diseased human brains.

### Example 8: Use of Micro-Radiobinding Assay to Determine Test Ligand Inhibitory Constant (Ki)

The micro-radiobinding assay was used to determine the inhibitory constant (Ki) of test ligands for AD-brain-derived tau deposits/aggregates and PD brain-derived a-syn deposits/aggregates. Proteins were prepared and spotted on coated glass slides as described in Examples 1 (steps a and b) and 2(a).

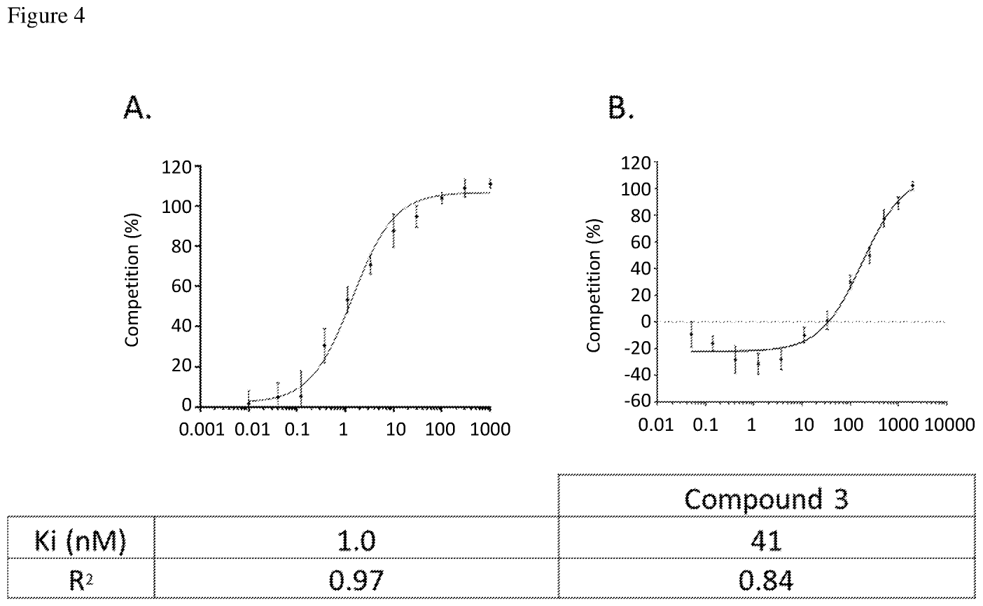

Tritiated test ligand (a known Tau binder) at 3 nM was incubated with spotted tau deposits/aggregates and cold test ligand at concentrations from 10 pM to 3 μM (FIG. 4A). Maximal signal (100% binding) was obtained in absence of cold tool ligand while maximal displacement was obtained in the presence of 3 μM of cold tool ligand. The Ki value was calculated by One site—Fit Ki using Prism V7 (GraphPad). The Ki value for the test ligand was measured at 1 nM with a good fitting of R²=0.97.

Cold Compound 3 was incubated with spotted a-syn deposits/aggregates at a range of concentrations from 50 pM to 2 μM (or 10 nM to 3 μM) along with 40 nM [3H] Compound 3. Maximal signal (100% binding) was obtained in absence of cold Compound 3 while maximal displacement was obtained in the presence of 2 μM of Compound 3. The Ki value for Compound 3 was measured at 41 nM with a good fit (FIG. 4B).

These results demonstrate that the self-displacement ability of a compound (determined by calculated Ki) can be determined by the described micro-radiobinding assay on a several protein targets that are known to be present in biological tissues in low or relatively low abundance.

### Example 9: Micro-Radiobinding Assay for Ranking Activity of Library Compounds

Test compounds were screened for their potency to compete with the binding of [3H]Compound 3 (radiolabeled tool ligand) to PD patient brain-derived a-syn deposits/aggregates. Test compound displacement was assessed in a screening format to allow ranking of test compounds based on their abilities to displace the radiolabeled tool ligand (ranking based on the calculated Ki values). Preparation and spotting of the protein samples were performed as described above in Examples 1(b) and 2(a).

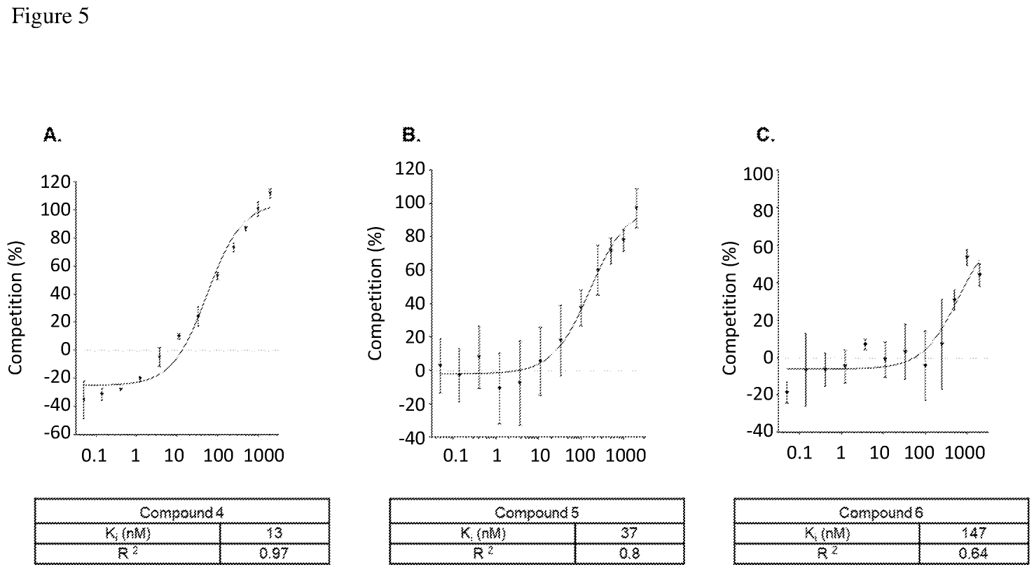

The test compounds were tested in two independent experiments in duplicates, with mean values±SEM shown in FIGS. 5A-5C. The test compounds were screened at concentrations ranging from 50 μM to 2 μM using [3H] Compound 3 at 40 nM as the radiolabeled tool ligand. Representative competition curves are shown for the following compounds: Compound 4 (FIG. 5A, Ki 13 nM, strong binder), Compound 5 (FIG. 5B, Ki 37 nM, intermediate binder), and Compound 6 (FIG. 5C, Ki 147 nM, weak binder). Taken together, these results demonstrate that the described micro-radiobinding assay can be used to measure displacement abilities (Ki) of test compounds in a screening format, which allows ranking of the screened test compounds according to the calculated Ki values, e.g., from the weakest to strongest binders. The test compounds showing the lower Ki values are considered as stronger binders and would represent potential hit compounds towards the tested protein target. In addition, the ability of a test compound to displace a radiolabeled tool ligand indicates that the test compound binds to the protein target at a site that overlaps with the protein binding site of the radiolabeled tool ligand.

While aspects of the present disclosure have been shown and described herein, it will be apparent to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the disclosure. It is intended that the following claims define the scope of the disclosure and that methods and structures within the scope of these claims and their equivalents be covered thereby.

### REFERENCES

Greenberg, S. G. and P. Davies, “A preparation of Alzheimer paired helical filaments that displays distinct tau proteins by polyacrylamide gel electrophoresis,” Proc. Natl. Acad. Sci. USA 1990, 87(15), 5827-31.
Goedert, M. et al., “Cloning of a big tau microtubule-associated protein characteristic of the peripheral nervous system,” Proc. Natl. Acad. Sci. USA 1992, 89, 1983-1987.
Jicha, G. A. et al., “A Conformation- and Phosphorylation-Dependent Antibody Recognizing the Paired Helical Filaments of Alzheimer's Disease,” J. Neurochem. 1997, 69, 2087-2095.
Mandelkow, E. and E. Mandelkow, “Tau in Alzheimer's disease,” Trends Cell Biol, 8(11), 425-427.
Neumann, M. et al., “Ubiquitinated TDP-43 in Frontotemporal Lobar Degeneration and Amyotrophic Lateral Sclerosis,” Science 2006, 314 (5796), 130-133.
Nelson, P. T. et al., “Limbic-predominant age-related TDP-43 encephalopathy (LATE): consensus working group report,” Brain 2019, 142(6), 1503-27.
Posner, B. et al., “Multiplexing G protein-coupled receptors in microarrays: A radioligand-binding assay,” Anal. Biochem. 2007, 365, 266-73.
Rostagno, A. and J. Ghiso, “Isolation and biochemical characterization of amyloid plaques and paired helical filaments,” Curr. Protoc. Cell Biol. 2009, 44(1), 3.33.1-3.33.33.
Spillantini, M. G. et al., “a-Synuclein in filamentous inclusions of Lewy bodies from Parkinson's disease and dementia with Lewy bodies,” Proc. Natl. Acad. Sci. USA 1998, 95, pp. 6469-6473.
Serrano-Pozo et al., “Neuropathological alternations in Alzheimer disease,” Cold Spring Harb. Perspect. Med. 2011, 1, a006189.

What is claimed is:

1. A method of determining binding affinity (K_{d}) of a test ligand for a pathological protein in an enriched biological sample comprising:
contacting a plurality of aliquots of the enriched biological sample on a microarray with a cold test ligand at saturating fixed concentration;
contacting the aliquots with a radiolabeled test ligand at multiple concentrations to form a radiolabeled complex between the radiolabeled test ligand and the pathological protein in each aliquot;
removing unbound radiolabeled test ligand from the aliquots;
detecting a signal from the radiolabeled test ligand in the radiolabeled complex in each aliquot; and
calculating the K_{d} from the detected signals in each aliquot.

2. A method of determining binding affinity (K_{d}) of a test ligand for a pathological protein in an enriched biological sample comprising:
contacting a plurality of aliquots of the enriched biological sample on a microarray with a radiolabeled test ligand at multiple concentrations to form a radiolabeled complex between the radiolabeled test ligand and the pathological protein in each aliquot;
contacting the aliquots with a cold test ligand at saturating fixed concentration;
removing unbound radiolabeled test ligand from the aliquots;
detecting a signal from the radiolabeled test ligand in the radiolabeled complex in each aliquot; and
calculating the K_{d} from the detected signals in each aliquot.

3. A method of determining binding affinity (K_{d}) of a test ligand for a pathological protein in an enriched biological sample comprising:
contacting a plurality of aliquots of the enriched biological sample on a microarray with a radiolabeled test ligand at multiple concentrations and a cold test ligand at saturating fixed concentration to form a radiolabeled complex between the radiolabeled test ligand and the pathological protein in each aliquot;
removing unbound radiolabeled test ligand from the aliquots;
detecting a signal from the radiolabeled test ligand in the radiolabeled complex in each aliquot; and
calculating the K_{d} from the detected signals in each aliquot.

4. A method of determining the inhibitory constant (Ki) of a test ligand for a pathological protein in an enriched biological sample comprising:
contacting a plurality of aliquots of the enriched biological sample on a microarray with a radiolabeled test ligand at a fixed concentration close to the K_{d} for the test ligand to form a radiolabeled complex between the radiolabeled test ligand and the pathological protein in each aliquot;
contacting the aliquots with a cold test ligand at multiple concentrations;
removing unbound radiolabeled test ligand from the aliquots;
detecting a signal from the radiolabeled test ligand in the radiolabeled complex in each aliquot; and
calculating the Ki from the detected signals in each aliquot.

5. A method of determining the inhibitory constant (Ki) of a test ligand for a pathological protein in an enriched biological sample comprising:
contacting a plurality of aliquots of the enriched biological sample on a microarray with a cold test ligand at multiple concentrations;
contacting the aliquots with a radiolabeled test ligand at a fixed concentration close to the K_{d} for the test ligand to form a radiolabeled complex between the radiolabeled test ligand and the pathological protein in each aliquot;
removing unbound radiolabeled test ligand from the aliquots;
detecting a signal from the radiolabeled test ligand in the radiolabeled complex in each aliquot; and
calculating the Ki from the detected signals in each aliquot.

6. A method of determining the inhibitory constant (Ki) of a test ligand for a pathological protein in an enriched biological sample comprising:
contacting a plurality of aliquots of the enriched biological sample on a microarray with a radiolabeled test ligand at a fixed concentration close to the K_{d} for the test ligand and a cold test ligand at multiple concentrations to form a radiolabeled complex between the radiolabeled test ligand and the pathological protein in each aliquot;
removing unbound radiolabeled test ligand from the aliquots;
detecting a signal from the radiolabeled test ligand in the radiolabeled complex in each aliquot; and
calculating the Ki from the detected signals in each aliquot.

7. A method of evaluating a test compound for the ability to displace a radiolabeled tool ligand in a radiolabeled complex with a pathological protein in an enriched biological sample comprising:
contacting a plurality of aliquots of the enriched biological sample on a microarray with a radiolabeled tool ligand at a fixed concentration close to the K_{d} of the tool ligand to form a radiolabeled complex between the radiolabeled tool ligand and the pathological protein in each aliquot;
contacting the aliquots with a cold test compound at a single concentration or at multiple concentrations;
removing unbound radiolabeled tool ligand from the aliquots;
detecting a signal from the radiolabeled tool ligand in the radiolabeled complex in each aliquot; and
calculating (a) the percent of competition for the cold test compound from the detected signals in each aliquot, where the cold test compound is contacted at a single concentration; or (b) the Ki for the cold test compound from the detected signals in each aliquot, where the cold test compound is contacted at multiple concentrations.

8. A method of evaluating a test compound for the ability to displace a radiolabeled tool ligand in a radiolabeled complex with a pathological protein in an enriched biological sample comprising:
contacting a plurality of aliquots of the enriched biological sample on a microarray with a cold test compound at a single concentration or at multiple concentrations;
contacting the aliquots with a radiolabeled tool ligand at a fixed concentration close to the K_{d} of the tool ligand to form a radiolabeled complex between the radiolabeled tool ligand and the pathological protein in each aliquot;
removing unbound radiolabeled tool ligand from the aliquots;
detecting a signal from the radiolabeled tool ligand in the radiolabeled complex in each aliquot; and
calculating (a) the percent of competition for the cold test compound from the detected signals in each aliquot, where the cold test compound is contacted at a single concentration; or (b) the Ki for the cold test compound from the detected signals in each aliquot, where the cold test compound is contacted at multiple concentrations.

9. A method of evaluating a test compound for the ability to displace a radiolabeled tool ligand in a radiolabeled complex with a pathological protein in an enriched biological sample comprising:
contacting a plurality of aliquots of the enriched biological sample on a microarray with a cold test compound at a single concentration or at multiple concentrations and with a radiolabeled tool ligand at a fixed concentration close to the K_{d} of the tool ligand to form a radiolabeled complex between the radiolabeled tool ligand and the pathological protein in each aliquot;
removing unbound radiolabeled tool ligand from the aliquots;
detecting a signal from the radiolabeled tool ligand in the radiolabeled complex in each aliquot; and
calculating (a) the percent of competition for the cold test compound from the detected signals in each aliquot, where the cold test compound is contacted at a single concentration; or (b) the Ki for the cold test compound from the detected signals in each aliquot, where the cold test compound is contacted at multiple concentrations.

10. The method of any one of claims 7 to 9, comprising:
(a) contacting the aliquots with multiple cold test compounds each at a single concentration, or
(b) contacting the aliquots with multiple cold test compounds at multiple concentrations.

11. The method of claim 10, comprising ranking the multiple test compounds according to the calculated percent of competition or Ki for each test compound.

12. The method of any one of claims 1 to 11, comprising contacting the aliquots of the enriched biological sample with a blocking agent.

13. The method of claim 12, wherein the aliquots are contacted with the blocking agent prior to or simultaneously with the radiolabeled tool ligand, cold tool ligand, and/or cold test compound.

14. The method of claim 13, wherein the blocking agent is present in one or more assay buffers that also comprise the radiolabeled tool ligand, cold tool ligand, or cold test compound.

15. The method of claim 14, wherein the assay buffer comprises Tris-HCl or phosphate-buffered saline (PBS).

16. The method of any one of claims 12 to 15, wherein the blocking agent is BSA, casein, or albumin from chicken egg white.

17. The method of any one of claims 1 to 16, wherein the pathological protein is selected from Abeta, Tau, α-synuclein, and TDP-43.

18. The method of any one of claims 1 to 17, wherein each aliquot of the enriched biological sample comprises from about 3.5 to about 6.5 mg/mL of total protein.

19. The method of any one of claims 1 to 18, wherein each aliquot is a spot.

20. The method of any one of claims 1 to 19, wherein the microarray comprises at least 5, or at least 10, or at least 15, or at least 20, or at least 25, or at least 30, or at least 40, or at least 45, or at least 50, or at least 55, or at least 60, or about 64 chambers.

21. The method of claim 20, wherein each chamber comprises at least one aliquot, or at least six aliquots, or at least nine aliquots, or nine aliquots of the enriched biological sample.

22. The method of claim 20 or claim 21, wherein contacting the aliquots with multiple concentrations comprises contacting each chamber in the microarray with a different concentration.

23. The method of any one of claims 1 to 22, wherein the detecting comprises detecting the signal on a film after exposing the microarray to the film.

24. The method of claim 23, wherein the film is a phosphoscreen film.

25. The method of any one of claims 1 to 24, wherein the microarray is a glass slide.

26. The method of claim 25, wherein the glass slide is an aminopropylsilane-coated glass slide.

27. The method of claim 25 or claim 26, comprising preparing the microarray by dispensing aliquots of the enriched biological sample onto the glass slide.

28. The method of any one of claims 1 to 27, comprising drying the plurality aliquots on the microarray before contacting with a radiolabeled test ligand or with a cold test ligand or compound.

29. The method of any one of claims 1 to 28, comprising drying before detecting a signal.
